(19) United States
(12) Patent Application Publication
(10) Pub. No.: US 2015/0209069 A1
(43) Pub. Date: Jul. 30, 2015

(54) Scalpel

(71) Applicant: Chris SALVINO, (US)

(72) Inventor: Chris Salvino, Sottsdale, AZ (US)

(21) Appl. No.: 14/424,587

(22) PCT Filed: Aug. 27, 2013
(86) PCT No.: PCT/US13/56834

§ 371 Date: Feb. 27, 2015

Related U.S. Application Data
(60) Provisional application No. 61/693,453, filed on Aug. 27, 2012.

(51) Int. Cl. A61B 17/3213

(52) U.S. Cl. CPC A61B 17/3213; A61B 2017/00792

(57) ABSTRACT

A scalpel for cutting soft tissues of the body with an abrasive trailing surface. The abrasive trailing surface reduces distinct scar formation by supporting wound healing. The scalpel can be mounted upon a traditional handle. It may also be implemented as a reciprocating saw, as a rotary drill and reciprocating saw, or as a circular and reciprocating saw.

### CROSS REFERENCE TO RELATED APPLICATIONS

This application claims priority to PCT Application number PCT/US13/056834 filed on Aug. 27, 2013 and U.S. provisional application No. 61/693,453 dated Aug. 28, 2012 the contents of which are hereby incorporated by reference.

### BACKGROUND

The United States medical industry uses tens of millions of scalpels per year. Most are made from high grade stainless steel and are used in a traditional handle. There has been very little change in stainless steel scalpels in form, function or results for more than a century. Other types of cutting devices such as laser and plasma beams, electric current cutting tools, ultrasonic knives, cryogenic knives, and high speed water jets have enjoyed varying degrees of success. But hand held stainless steel scalpels are still the most commonly used cutting tool. Unfortunately stainless steel scalpels produce unsatisfactory cosmetic results for two reasons: the formation of conspicuous scars and beveling in curved incisions.

Distinct scars form because the leading edge of the scalpel is too sharp. When the skin is cut with a modern scalpel the surface of the tissue on either side of the incision is extremely smooth and uniform. In addition, just a few cell layers below the cut surface the tissue is unaffected. As a result the area in which healing takes place is highly concentrated resulting in a conspicuous scar zone having a “welded” appearance.

Beveling occurs with any curved incision because the length of the cutting edge is much larger than the linear distances found within sharp corners. As a result the cutting edge tends to “skid” as it is drawn around an acute angle resulting in uneven and excessive cutting which leads to greater scarring.

### PRIOR ART REFERENCES

U.S. Pat. No. 7,842,058 issued to Simpson discloses a powered scalpel that gives both slicing and downward cutting actions.

United States patent application number 2010/0087845 A1 issued to Spiro discloses an extremely sharp blade with specified tolerances of 4 um or less.

U.S. Pat. No. 4,887,598 issued to Berke discloses a manual rotary scalpel structure with a bifurcated end so that the blade can be rotably mounted.

United States patent applicaiton number 2012/0029545 A1 issued to Nelson discloses a reciprocating surgical instrument with a tissue suction element.

U.S. Pat. No. 5,441,512 issued to Muller discloses a high incision velocity vibrating scalpel structure using a traditional linear scalpel blade mounted on the end of a cylindrical handle producing an essentially arcuate movement.

U.S. Pat. No. 8,136,251 issued to Endo discloses a medical edged tool having an accommodated state in which the blade is hidden, and a projected state where it sticks out.

U.S. Pat. No. 8,162,961 issued to Zaporojan discloses a device and method for cutting and suturing biological tissue, primarily for meniscus of the knee.

U.S. Pat. No. 8,187,294 issued to Miller discloses a rotating scalpel device using inner and outer cannula.

### FIELD OF THE INVENTION

The present invention is in the field of medical devices and pertains to cutting, severing or perforating soft tissues of the body in operative surgery.

### SUMMARY OF THE INVENTION

In view of the shortcomings of the prior art, it is the object of this invention to provide an improved scalpel which avoids the problem of distinct scar formation and can produce incisions that heal with less conspicuous scars and are more cosmetically pleasing.

Another object of the present invention is to provide an improved scalpel which avoids the problem of beveling and can produce curved cuts that are precise at every point along the curve.

Further objects and advantages of the invention will become apparent to those skilled in the art upon reading and consideration of the following description of a preferred embodiment and the accompanying drawings.

### BRIEF DESCRIPTION OF THE DRAWINGS

FIG. 1 shows a preferred embodiment of a scalpel where the trailing surface is abrasive and the blade is adapted to be mounted upon a handle according to the invention.

FIG. 2 shows a top down view of the scalpel in FIG. 1 where the abrasive trailing surface is also wider than the leading edge according to the invention.

FIG. 3 shows a preferred embodiment of a scalpel where the leading edge of the blade is square, has an abrasive trailing surface and is adapted to be mounted upon a reciprocating saw according to the invention.

FIG. 4 shows preferred embodiments of a scalpel where the leading edge of the blade is rounded (4A) or angled (4B), has an abrasive trailing surface and is adapted to be mounted upon a reciprocating saw according to the invention.

FIG. 5 shows a preferred embodiment of a scalpel where the leading edge is tapered to a point and is followed by an abrasive trailing surface and is also disposed with both rotary and reciprocating motion according to the invention.

FIG. 6 shows a preferred embodiment of a scalpel where the leading edge is upon the outer edge of a circular blade with a wider, abrasive inner surface and is also disposed with both rotary and reciprocating motion according to the invention.

### DETAILED DESCRIPTION OF THE EMBODIMENTS

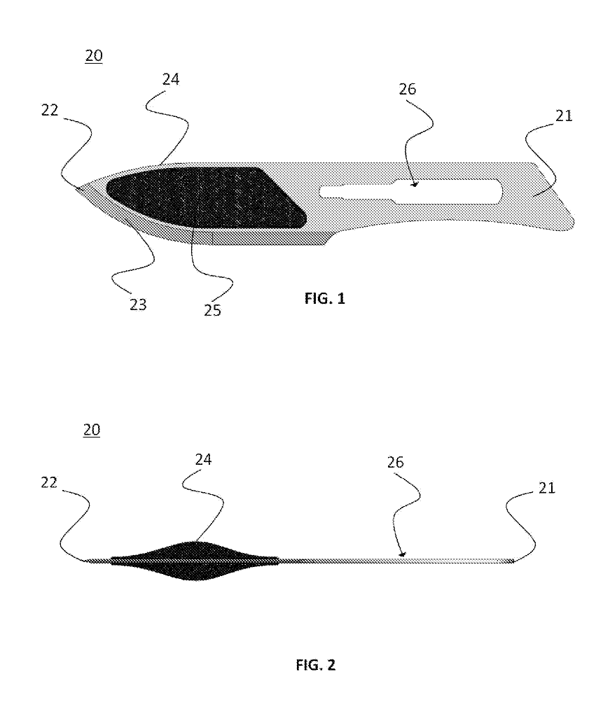

Referring now to the drawings wherein the showings are for purposes of illustrating a preferred embodiment of the present invention and not for purposes of limiting the same A first embodiment of the invention is a stainless steel scalpel 20 illustrated in FIG. 1 with a proximal end 21, a distal end 22, a leading or anterior edge 23, and a trailing or posterior edge 24. Scalpel 20 is disposed with an abrasive trailing surface 25 that occupies most of the distal 22 half of the blade immediately posterior to the leading edge 23. The abrasive trailing surface 25 is formed by grinding or blasting the blade with another abrasive material such as aluminum oxide or ceramic aluminum oxide. The abrading surface can also be formed by accretion in which grit particles made from steel or composite material are bonded and then scintered to the blade. The abrasive surface may be composed of peaks or valleys or both. It may be composed of conical, pyramidal or spherical protrusions which may be uniformly or irregularly distributed upon the trailing surface of the blade. The average extent of protrusion from the top of a peak or bottom of a valley may be between 10-1200 micrometers from the plane of the leading or cutting edge, preferably between 20-200 micrometers. A top down view of the scalpel is illustrated in FIG. 2 where the abrasive trailing edge 24 is also wider than the leading edge. The increase in thickness is approximately normally distributed over the distal 22 to proximal 21 length of the cutting edge, and is widest directly behind the midpoint of the cutting edge. In the present embodiment the trailing surface is significantly wider than the leading edge, but in other embodiments may be less wide depending upon the abrasive surface. Scalpel 20 is adapted to be mounted upon a handle 26 and is sized according to standard scalpel sizes. In this static embodiment of the scalpel blade, cutting is achieved by pulling or pushing the distal-anterior blade against the tissue in the x-axis

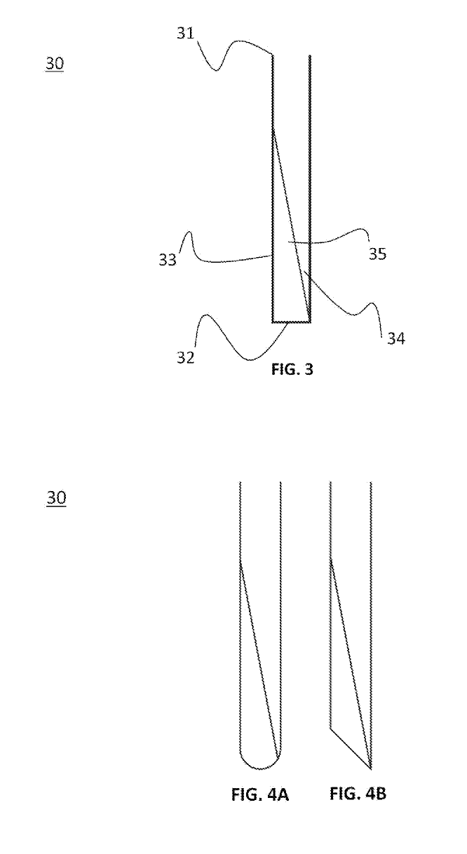

A second embodiment of the invention is a stainless steel scalpel 30, illustrated in FIG. 3, with a proximal end 31, a distal end 32, a leading or anterior edge 33, and a trailing or posterior edge 34. Both the distal end 32 and the leading edge 33 are cutting surfaces. Scalpel 30 is disposed with an abrasive trailing surface 35 that occupies most of the distal 32 and posterior 34 area of the cutting surface. Scalpel 30 is between 50-500 mm in length, but has a very narrow distal edge width 32. Scalpel 30 is adapted to be mounted upon a reciprocating saw at the proximal end 31. In this embodiment, the anterior edge 33 and posterior surface 34 meet at the distal end 32 so that the tip forms a square edge. In other embodiments illustrated in FIG. 4, the anterior and posterior edges may meet so that they form a rounded, FIG. 4A, or angled FIG. 4B, distal edge. In this dynamic embodiment of the reciprocating scalpel blade cutting is achieved by pushing or pulling the blade against the tissue in the x-axis and the automatic reciprocating motion in the y-axis. The angle that defines the edge between anterior edge 33 and distal end 32 may vary depending on the dimensions of the particular blade, for example, it is preferable that the leading edge cut at least 1 mm of tissue before the posterior surface 34 contacts the tissue.

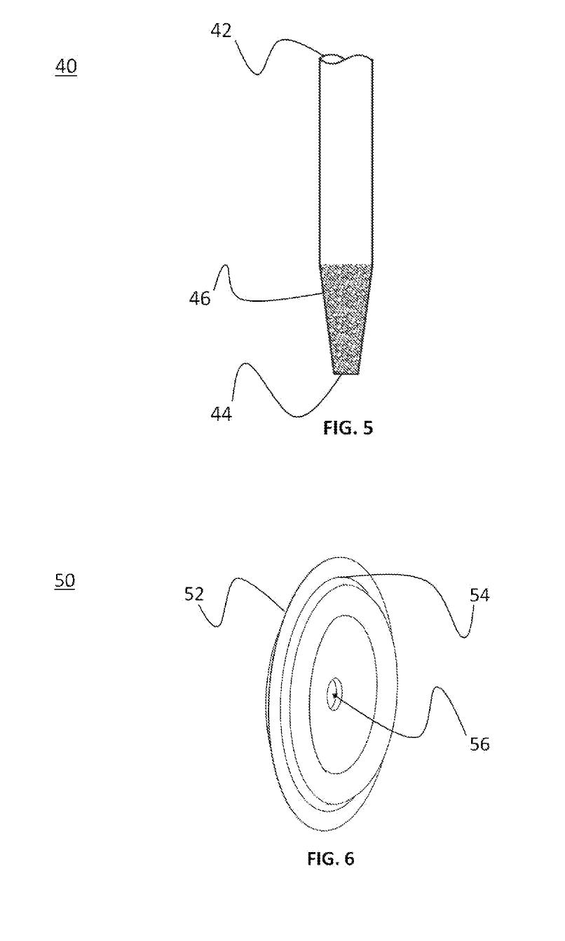

A third embodiment of the invention is illustrated in FIG. 5, a cylindrical stainless steel scalpel 40, with a proximal end 42, a distal end 44 that is tapered. Scalpel 40 is disposed with an abrasive trailing surface 46 that is located on the tapered portion proximal to the distal tip 44. In the present embodiment, scalpel 40 is a solid cylinder with an abrading surface on the outside only, but in other embodiments it may be a hollow cylinder with an open tip having abrading surfaces on either the outside or the inside or both surfaces of the cylinder. Scalpel blade 40 is between 10-200 mm in length, but has a very narrow diameter even on the un-tapered portion of the cylinder, preferably 0.1-2.0 mm. The proximal end 42 is adapted to be mounted within a drill bit that imparts both rotary and reciprocating motion. In this dynamic embodiment of the reciprocating scalpel blade cutting is achieved by pushing or pulling the blade against the tissue in the x-axis and the automatic reciprocating motion in the y-axis.

A fourth embodiment of the invention is a circular stainless steel scalpel 50 illustrated in FIG. 6. with a leading or outer edge 52, an abrasive trailing or inner edge 54. The center of the circular blade is also adapted to be mounted upon a rotary saw 56. Scalpel 50 is between 10-100 mm in diameter. In this dynamic embodiment of the circular scalpel cutting is achieved by pushing or pulling the spinning blade against the tissue in the x-axis. In another embodiment scalpel 50 would have both rotary and reciprocating motion. In this dynamic embodiment of the reciprocating scalpel blade cutting is achieved by pushing or pulling the blade against the tissue in the x-axis and the automatic reciprocating motion in the y-axis.

While the embodiments have been described as being made of stainless steel other surgical cutting materials are known such as cobalt chromium.

Methods of scintering stainless steel and cobalt chromium are known arts essentially comprising a mixture of metal shot, an adhesive, and a sacrificial material. The mixture is applied to a scalpel and heated up to evaporate the sacrificial material (1000 degrees Celcius) and then heated to a higher temperature (1600 degrees Celsius) to scinter the metal shot to the surface of the scalpel. Scintering can be done in various gas and under various pressures depending on the particular metal shot.

Methods of abraiding a metal surface are also known in the arts. For example, a scalpel could have the sharp leading edge masked with a protective wax or tape and the trailing surface of the scalpel could be sand blasted to remove material. The wax or tape could be removed and the scalpel could be cleaned and sterilized using commercially available methods.

Additional modifications and improvements of the present invention may also be apparent to those skilled in the art. Thus, the particular combination of parts described and illustrated herein in intended to represent only one embodiment of the invention, and is not intended to serve as limitations of alternative devices within the spirit and scope of the invention.

What is claimed is:

1. A scalpel for cutting, severing, or perforating soft tissues of the body in operative surgery comprising an abrasive trailing surface

2. The scalpel of claim 1 where the scalpel is produced from materials chosen from the group consisting of stainless steel, steel alloys and composite ceramics.

3. The scalpel of claim 1 where the abrading surface is formed by grinding or blasting the blade with another abrasive material

4. The scalpel of claim 1 where the abrading surface is formed by accretion in which grit particles made from steel or composite material are bonded and then sintered to the blade

5. The scalpel of claim 1 where the abrading surface is disposed with an average extent of protrusion from the plane of the leading or cutting edge by 10-1200 micrometers

6. The scalpel of claim 1 where the scalpel is mounted upon a handle.

7. The scalpel of claim 1 where the abrading surface is located upon the distal half of the blade and extends from the posterior edge anteriorly to the leading edge.

8. The scalpel of claim 1 where the trailing edge is wider than the leading edge and the increase in thickness is approximately normally distributed over the distal to proximal length of the blade,

9. The scalpel of claim 1 where the distal tip is squared.

10. The scalpel of claim 1 where the distal tip is rounded.

11. The scalpel of claim 1 where the distal tip is angled.

12. The scalpel of claim 1 where the abrading surface is located behind both the leading edge and distal tip cutting surfaces.

13. The scalpel of claim 1 wherein the scalpel is mounted upon a reciprocating saw.

14. The scalpel of claim 1 where the blade is cylindrical and has a tip tapered to a point.

15. The scalpel in claim 1 where the cylindrical blade is solid.

16. The scalpel in claim 1 where the cylindrical blade is hollow.

17. The scalpel in claim 1 where the abrading surface is located behind the tapered tip.

18. The scalpel in claim 1 wherein the abrading surface is on the outside of the cylindrical blade.

19. The scalpel in claim 1 wherein the abrading surface is on the inside of the cylindrical blade.

20. The scalpel in claim 1 where the abrading surface is on both the inside and the outside of the cylindrical blade.

21. The scalpel of claim 1 where the blade is circular.

22. The scalpel of claim 1 where the abrading surface is located behind the leading edge and extends towards the center of the circular blade

23. The Method of Using the Scalpel of claim 1.

24. The Method of making the Scalpel of claim 1.
